(19) United States
(12) Patent Application Publication
(10) Pub. No.: US 2025/0123346 A1
(43) Pub. Date: Apr. 17, 2025

(54) SYSTEM AND METHOD FOR PATIENT SPECIFIC MAGNETIC RESONANCE IMAGING PROTOCOL ADJUSTMENTS TO OBTAIN EXPECTED MAGNETIC RESONANCE IMAGE QUALITY

(71) Applicant: GE Precision Healthcare LLC, Waukesha, WI (US)

(72) Inventors: Tisha Anie Abraham, Bangalore (IN); Dattesh Dayanand Shanbhag, Bangalore (IN); Harsh Kumar Agarwal, Jaipur (IN); Sheila Srinivasan Washburn, Brookfield, WI (US); Maggie MeiKei Fung, Jersey City, NJ (US); Suchandrima Banerjee, Berkeley, CA (US); Patrick Quarterman, New York, NY (US); Ramesh Venkatesan, Bangalore (IN); Sajith Rajamani, Bangalore (IN)

(21) Appl. No.: 18/484,860

(22) Filed: Oct. 11, 2023

(51) Int. Cl. G01R 33/54; A61B 5/00; A61B 5/055

(52) U.S. Cl. CPC G01R 33/543; A61B 5/055; A61B 5/7203; A61B 5/7485

(57) ABSTRACT

A method includes receiving a selection of a scan protocol for the scan of a subject and obtaining localizer images including an anatomic landmark of interest of the subject acquired with the MRI system. The method includes automatically detecting the anatomic landmark of interest in localizer images and determining a geometry plan of the scan including extents of the anatomic landmark of interest. The method includes automatically determining a coverage of the scan to include the anatomic landmark of interest and to match the extents of the anatomic landmark of interest. The method includes obtaining limits on adjustments scan time and one or more image quality parameters for the scan protocol. The method includes generating an updated scan protocol by automatically adjusting one or more parameters of the scan protocol based on the scan protocol, the limits on adjustments, and the coverage of the scan.

### BACKGROUND

The subject matter disclosed herein relates to medical imaging and, more particularly, to a system and a method for patient specific magnetic resonance imaging (MRI) protocol adjustments to obtain expected MR image quality.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, Mz, may be rotated, or “tipped”, into the x-y plane to produce a net transverse magnetic moment, Mt. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Each MRI site defines a set of protocols for MR data acquisition which would yield certain quality MR images for a typical patient size. Intelligent prescription for a scan of a specific subject provides the desired geometry plan for the scan but disregards information regarding a patient's size. This is done to ensure image quality metrics related to a site protocol are maintained. While this approach ensured that image quality was not disturbed, scan time for a scan would vary based on the size of the patient. In addition, for a specific subject's scan, the MR technologist invariably changes parameters related to the scan protocol which also changes image quality metrics (e.g., signal-to-noise ratio, contrast-to-noise ratio, etc.).

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing a scan of a subject utilizing a magnetic resonance imaging (MRI) system is provided. The computer-implemented method includes receiving, at a processor, a selection of a scan protocol from among a plurality of scan protocols for the scan of the subject with the MRI system. The computer-implemented method also includes obtaining, at the processor, localizer images including an anatomic landmark of interest of the subject acquired with the MRI system. The computer-implemented method further includes automatically detecting, via the processor, the anatomic landmark of interest in the localizer images and determining a geometry plan of the scan of the anatomic landmark of interest including extents of the anatomic landmark of interest of the subject based on the localizer images. The computer-implemented method even further includes automatically determining, via the processor, a coverage of the scan to include the anatomic landmark of interest and to match the extents of the anatomic landmark of interest based on the scan protocol and the geometry plan including the extents of the anatomic landmark of interest. The computer-implemented method yet further includes obtaining, at the processor, limits on adjustments of scan time and one or more image quality parameters for the scan protocol. The computer-implemented method still further includes generating, via the processor, an updated scan protocol by automatically adjusting one or more parameters of the scan protocol based on the scan protocol, the limits on adjustments, and the coverage of the scan.

In another embodiment, a system for performing a scan of a subject utilizing an MRI system is provided. The system includes a memory encoding processor-executable routines. The system also includes a processor configured to access the memory and to execute the processor-executable routines, wherein the routines, when executed by the processor, cause the processor to perform actions. The actions include receiving a selection of a scan protocol from among a plurality of scan protocols for the scan of the subject with the MRI system. The actions also include obtaining images including an anatomic landmark of interest of the subject acquired with the MRI system, wherein the images have a higher resolution than localizer images. The actions further include automatically detecting the anatomic landmark of interest in the images and determining a geometry plan of the scan of the anatomic landmark of interest including extents of the anatomic landmark of interest of the subject based on the images. The actions even further include automatically determining a coverage of the scan to include the anatomic landmark of interest and to match the extents of the anatomic landmark of interest based on the scan protocol and the geometry plan including the extents of the anatomic landmark of interest. The actions yet further include obtaining limits on adjustments of scan time one or more image quality parameters for the scan protocol. The actions still further include generating an updated scan protocol by automatically adjusting one or more parameters of the scan protocol based on the scan protocol, the limits on adjustments, and the coverage of the scan.

In a further embodiment, a non-transitory computer-readable medium, the computer-readable medium including processor-executable code that when executed by a processor, causes the processor to perform actions. The actions include receiving a selection of a scan protocol from among a plurality of scan protocols for a scan of a subject with a magnetic resonance imaging (MRI) system. The actions also include obtaining localizer images including an anatomic landmark of interest of the subject acquired with the MRI system. The actions further include automatically detecting the anatomic landmark of interest in the localizer images and determining a geometry plan of the scan of the anatomic landmark of interest including extents of the anatomic landmark of interest of the subject based on the localizer images. The actions even further include automatically determining a coverage of the scan to include the anatomic landmark of interest and to match the extents of the anatomic landmark of interest based on the scan protocol, the geometry plan including the extents of the anatomic landmark of interest. The actions yet further include obtaining limits on adjustments of scan time and one or more image quality parameters for the scan protocol. The actions still further include generating an updated scan protocol by automatically adjusting one or more parameters of the scan protocol based on the scan protocol, the limits on adjustments, and the coverage of the scan.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 illustrates a schematic diagram of a site having a plurality of MRI systems, in accordance with aspects of the present disclosure;

FIG. 3 illustrates a schematic diagram of a prescription engine utilized by an MRI system, in accordance with aspects of the present disclosure;

FIG. 4 illustrates a schematic diagram of a first module of the prescription engine 188 in FIG. 3, in accordance with aspects of the present disclosure;

FIG. 5 illustrates a flow diagram of a method for performing a scan of a patient utilizing the MRI system in FIG. 1, in accordance with aspects of the present disclosure;

FIG. 6 illustrates a schematic diagram of a workflow for performing a scan of a patient, in accordance with aspects of the present disclosure;

FIG. 7 is an MR image obtained of an anatomic landmark of interest (e.g., brain) of a subject;

FIG. 8 is an MR image obtained of an anatomic landmark of interest (e.g., brain) of a subject (e.g., taking into account patient size), in accordance with aspects of the present disclosure;

FIG. 9 illustrates a flow diagram of a method for performing a scan of a patient utilizing the MRI system in FIG. 1 (e.g., utilizing higher resolution images instead of scout or localizer images), in accordance with aspects of the present disclosure; and

FIG. 10 is a schematic diagram illustrating outputs of an auto-parameters module, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for improving prescription for an MRI scan of a subject. In particular, the systems and methods include receiving a selection of a scan protocol from among a plurality of scan protocols for the scan of the subject with the MRI system. A scan protocol prescribes a specific anatomical landmark (versus all landmarks possible), pulse sequence parameters (e.g., echo time (TE), repetition time (TR), matrix size, etc.), and other factors related to performing an MR scan. The systems and methods also include obtaining localizer images including an anatomic landmark of interest of the subject acquired with the MRI system. The systems and methods further include automatically detecting the anatomic landmark of interest in the localizer images and determining a geometry plan of the scan of the anatomic landmark of interest including extents of the anatomic landmark of interest of the subject based on the localizer images. In certain embodiments, the systems and methods include obtaining images (e.g., non-diagnostic images) including an anatomic landmark of interest of the subject acquired of the subject, wherein the images have a higher resolution than scout or localizer images. In certain embodiments, the systems and methods include automatically detecting the anatomic landmark of interest in the localizer images and determining a geometry plan of the scan of the anatomic landmark of interest including extends of the anatomic landmark of interest of the subject based on the images (e.g., higher resolution images).

The systems and methods even further include automatically determining a coverage of the scan to include the anatomic landmark of interest and to match the extents of the anatomic landmark of interest based on the scan protocol and the geometry plan including the extents of the anatomic landmark of interest. The systems and methods yet further include obtaining limits on adjustments of scan time and one or more image quality parameters (e.g., image quality metrics) for the scan protocol. The systems and methods still further include generating an updated scan protocol by automatically adjusting one or more parameters of the scan protocol based on the scan protocol, the limits on adjustments, and the coverage of the scan.

In certain embodiments, the systems and methods include initiating the scan of the subject utilizing the updated scan protocol. In certain embodiments, the updated scan protocol maintains an image quality of an acquired image obtained from the scan with respect to a baseline protocol (i.e., the selected scan protocol). In certain embodiments, a contrast-to-noise ratio is maintained in the acquired image. In certain embodiments, the contrast is kept constant but the noise may be minimally adjusted, thus, slightly adjusting the contrast-to-noise ratio (but keeping it within a desired limit). In certain embodiments, both a signal-to-noise ratio and a contrast-to-noise ratio in the acquired image. In certain embodiments, the one or more adjusted parameters include a signal-to-noise ratio. In certain embodiments, the limits on adjustments apply to use of all MRI systems at a site having one or more MRI systems. A site may be hospital, MR imaging center, or other facility having one or more MRI systems. The site may be part of a network (e.g., hospital system) having multiple sites, where each site may have one or more MRI systems. Site specific configurations or limits may be network specific configurations or limits that may be the same across the entire network (i.e., all of the sites of the network). In certain embodiments, some sites of a network may not have an MRI system. In certain embodiments, the limits on adjustments are based on a condition of the subject. In certain embodiments, the one or more adjusted parameters includes matrix size (e.g., MatrixX and Matrix Y), echo train length, and/or an acceleration factor for parallel imaging.

The disclosed embodiments enable utilization of a prescription engine that utilizes patient size-related constraints while ensuring image quality does not differ from baseline protocol within site-specified limits. The disclosed embodiments enable restricting a scan to an anatomy of interest and avoiding scanning unwanted regions. The disclosed embodiments enable providing optimal image quality irrespective of patient size changes and ensure reduced scan time. The disclosed embodiments enable wider acceptance of intelligent prescription. The disclosed embodiments enable less dependence on an MR technologist's expertise to obtain quality MR mages at an MR center (i.e., avoiding the interaction of the technologist to update prescription parameters to optimize image quality and scan time). The disclosed embodiments enable providing consistent MR images of good quality across an MR scanner for a patient. The disclosed embodiments enable a site to configure the ranges of the parameter adjustments to their preference.

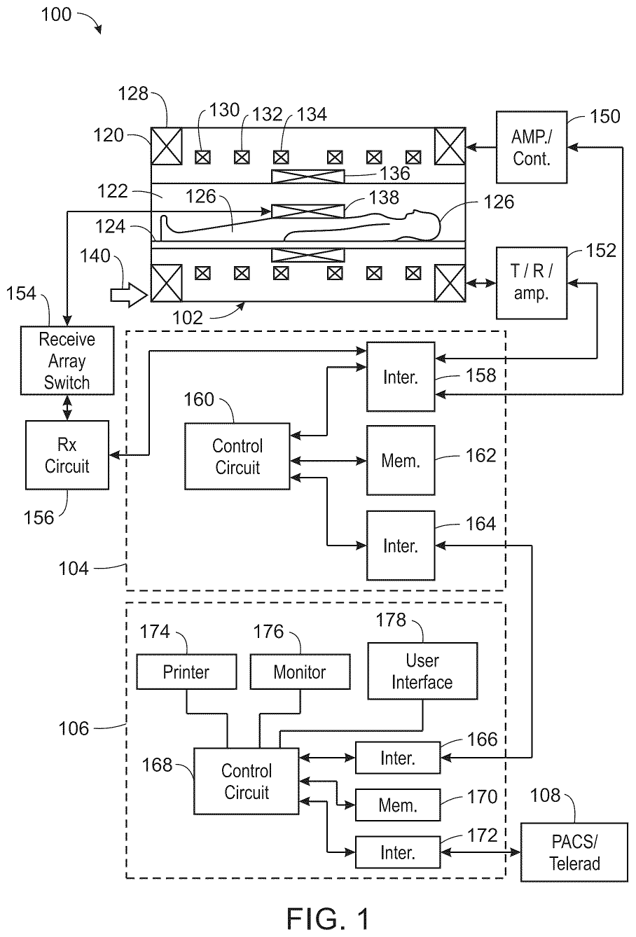

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, Bo. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural networks for prescribing parameters for a scan as described below. In certain embodiments, image reconstruction may occur on a separate computing device having processing circuitry and memory circuitry.

The programming code may enable receiving a selection of a scan protocol from among a plurality of scan protocols for the scan of the subject with the MRI system. The programming code may also enable obtaining localizer images including an anatomic landmark of interest of the subject acquired with the MRI system. The programming code may further enable automatically detecting the anatomic landmark of interest in localizer images and determining a geometry plan of the scan of the anatomic landmark of interest including extents of the anatomic landmark of interest of the subject based on the localizer images. The programming code may even further enable automatically determining a coverage of the scan to include the anatomic landmark of interest and to match the extents of the anatomic landmark of interest based on the scan protocol and the geometry plan including the extents of the anatomic landmark of interest. The programming may yet further enable obtaining limits on adjustments of scan time and one or more image quality parameters (e.g., image quality metrics) for the scan protocol. The programming code may still further enable generating an updated scan protocol by automatically adjusting one or more parameters of the scan protocol based on the scan protocol, the limits on adjustments, and the coverage of the scan.

In certain embodiments, the programming code may enable initiating the scan of the subject utilizing the updated scan protocol. In certain embodiments, the updated scan protocol maintains an image quality of an acquired image obtained from the scan with respect to a baseline protocol (i.e., the selected scan protocol). In certain embodiments, a contrast-to-noise ratio is maintained in the acquired image. In certain embodiments, the contrast is kept constant but the noise may be minimally adjusted, thus, slightly adjusting the contrast-to-noise ratio (but keeping it within a desired limit). In certain embodiments, both a signal-to-noise ratio and a contrast-to-noise ratio in the acquired image. In certain embodiments, the one or more adjusted parameters include a signal-to-noise ratio. In certain embodiments, the limits on adjustments apply to use of all MRI systems on a site (e.g., hospital, MR imaging center, etc.) having one or more MRI systems. In certain embodiments, the limits on adjustments are based on a condition of the subject. In certain embodiments, the one or more adjusted parameters includes matrix size (e.g., MatrixX and MatrixY), echo train length, and/or an acceleration factor for parallel imaging.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

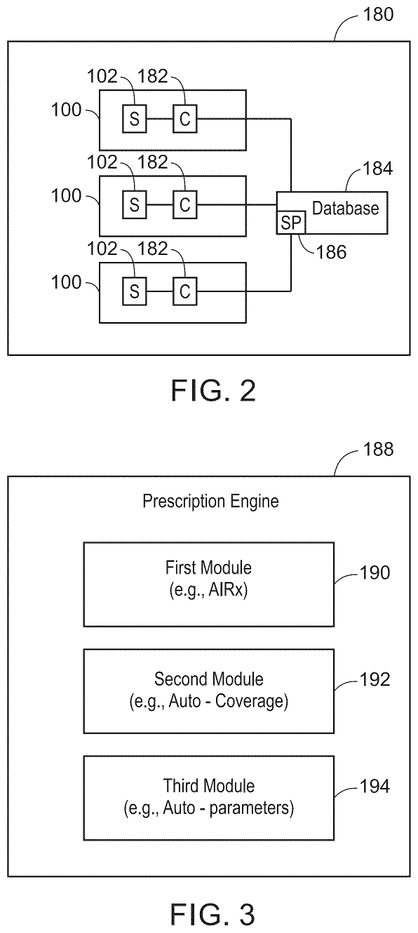

FIG. 2 illustrates a site 180 having a plurality of MRI systems 100. The site 180 may be a hospital, an MRI center, or any other medical facility that utilizes MRI systems 100. As depicted, the site 180 has multiple MRI systems 100. In certain embodiments, the site 180 may only have a single MRI system 100. As noted above, the site 180 may be part of a network (e.g., hospital system) having multiple sites 180, where each site 180 may have one or more MRI systems 100. Site specific configurations or limits may be network specific configurations or limits that may be the same across the entire network (i.e., all of the sites 180 of the network). In certain embodiments, some sites 180 of a network may not have an MRI system 100. Each MRI system 100 includes a respective MRI scanner 102 coupled to a respective control console 182 (e.g., operator console). Each control console 182 of a respective MRI system 100 is communicatively coupled to and enabled to access a database 184 storing a plurality of scan protocols 186 for utilization with the MRI systems 100. The plurality of scan protocols 186 are configured to be utilized across all of the MRI systems 100 for the site 180. Each scan protocol 186 is configured for an MRI data acquisition that yield an expected (e.g., certain) quality for MR images. Each scan protocol 186 is associated with site-specific limits (e.g., ranges of parameter adjustments) related to scan time and image quality parameters. The site-specific limits may be part of the selected scan protocol or obtained separately from the database 184. These site-specific limits may be modified for the site 180 via a remote computing device and/or one of the control consoles 182. Each control console 182 is utilized to select the desired scan protocol 186 from among the plurality of scan protocols 186. In addition, each control console 182 utilizes a prescription engine (e.g., intelligent prescription engine) configured to detect an anatomical landmark of interest and to prescribe a geometry plan for a scan (e.g., diagnostic MRI scan) utilizing the selected scan protocol. As described in greater detail below, the prescription engine may take into account the size of the patient (e.g., patient size-related constraints) in ensure that the scan is restricted to only the anatomy (anatomic landmark) of interest and avoids scanning unwanted regions. In addition, the prescription engine takes into account the site-specific limits in modifying prescription parameters of the scan protocol 186 to generate an updated scan protocol where image quality will be maintained (i.e., does not differ from the baseline protocol within the site-specified limits) irrespective of patient size.

FIG. 3 illustrates a prescription engine 188 utilized by an MRI system (e.g., MRI system 100 in FIG. 1). The prescription engine 188 is configure to provide an optimal image quality and optimal scan time for a specific subject and anatomical landmark give the extents of the anatomical landmark and a determined scan coverage based on the extents of the anatomical landmark. The prescription engine 188 includes a first module 190, a second module 192, and a third module 194. Each module 190, 192, and 194 may utilize one or more deep learning algorithms (and one or more trained neural networks) to perform their respective functions. The first module 190 (e.g., an intelligent prescription module such as AIRₓ™ from General Electric Healthcare) is configured to automatically detect anatomic landmark of interest in localizer images and determine a geometry plan (e.g., prescribed slices including center and orientation) of the scan of the anatomic landmark of interest including extents of the anatomic landmark of interest of the subject based on the localizer images. The second module 192 (e.g., auto-coverage module) is configured to automatically determine a coverage of a scan based on the scan protocol and the geometry plan (e.g., center and orientation) including the extents (and landmarks) of the anatomic landmark of interest. The third module 194 (e.g., auto-parameters module) is configured to obtain limits (site-specific configuration limits) on adjustments of a scan time and one or more image quality parameters. In certain embodiments, the limits on adjustments are based on a condition of the subject. The third module 194 is also configured to generate an updated scan protocol based on the scan protocol, the limits on adjustments, and the coverage of the scan.

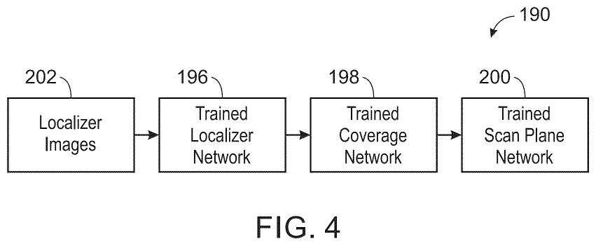

Each module 190, 192, and 194 may utilize one or more trained deep-learning based networks or models to perform various functions. For example, as depicted in FIG. 4, the first module 190 includes a trained localizer network 196, a trained coverage network 198, and a trained scan plane network 200. The trained localizer network 196 is configured to receive or obtain localizer or scout images 202 of a subject (e.g. patient) obtained from an MRI scanner. The localizer images 202 are a series of images (e.g., two-dimensional (2D) images) in the axial, sagittal, and coronal planes (e.g., 3-plane localizer scans). The localizer images 202 enable planning of the slice locations for higher resolution scans (e.g., diagnostic scans) of the patient. The trained localizer network 196 is configured to determine if a given localizer image 202 (e.g., coronal, axial, or sagittal) is suitable for identifying anatomic landmark of interest. If trained localizer network 196 determines that the obtained localizer images 202 are not suitable, then a prompt is provided to a technologist to repeat the scout scan. The trained coverage network 198 is configured to determine the extents (e.g., anterior-posterior (AP), right-left (RL), superior-inferior (SI)) of the anatomic landmark of interest in each localizer image 202. The trained scan plane network 200 is configured to determine the best orientation and location to target anatomic landmark of interest from the localizer images 202. In certain embodiments, the first module 190 may be utilized on obtained higher resolution images (e.g., axial images such as axial T2W images) of the subject. The higher resolution images have a higher resolution than scout or localizer images.

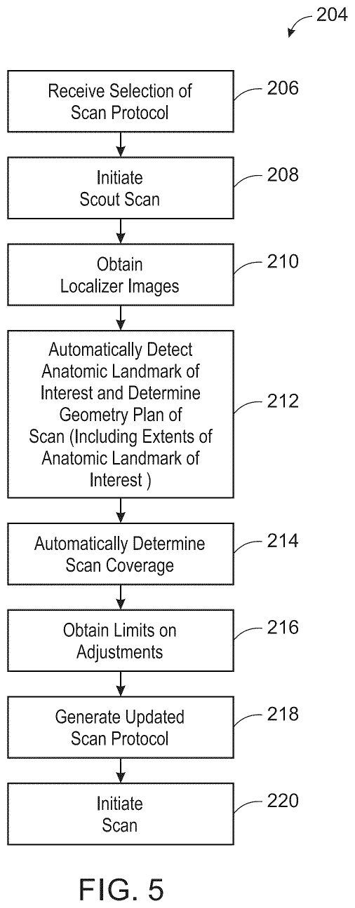

FIG. 5 illustrates a flow diagram of a method 204 for performing a scan of a patient utilizing the MRI system 100 in FIG. 1 utilizing an improved MR scanning workflow. One or more steps of the method 204 may be performed by processing circuitry of the magnetic resonance imaging system 100 in FIG. 1. One or more of the steps of the method 204 may be performed simultaneously or in a different order from the order depicted in FIG. 5.

The method 204 includes receiving a selection of a scan protocol from among a plurality of scan protocols for a scan of a subject (e.g., patient) with an MRI system (e.g., MRI system 100 in FIG. 1) (block 206). The method 204 also includes initiating a scout scan (e.g., non-diagnostic scan) of the subject with the MRI system to obtain localizer or scout images (block 208). The localizer images are a series of images (e.g., two-dimensional (2D) images) in the axial, sagittal, and coronal planes (e.g., 3-plane localizer scans). The method 204 further includes obtaining the localizer images including an anatomic landmark of interest of the subject (block 210).

The method 204 still further includes automatically detecting the anatomic landmark of interest in localizer images and determining a geometry plan (e.g., prescribed slices including center and orientation) of the scan of the anatomic landmark of interest including extents (e.g., AP, RL, SI) of the anatomic landmark of interest of the subject based on the localizer images (block 212). The first module 190 of the prescription engine 188 in FIG. 3 is utilized for block 212 of the method 204. The first module 190 also determines as part of the geometry plan a center and orientation. The first module 190 further determines landmarks of the anatomic landmark of interest.

The method 204 even further includes automatically determining a coverage of the scan to include the anatomic landmark of interest and to match the extents of the anatomic landmark of interest based on the scan protocol and the geometry plan, including the extents of the anatomic landmark of interest (block 214). The determined coverage restricts the scan to the anatomic landmark of interest and avoids scanning unwanted regions. The second module 192 of the prescription engine 188 in FIG. 3 is utilized for block 214 of the method 204. Determining the coverage includes determining and outputting one or more a number of slices, a field of view in the X direction (FOV X), and a field of view in the Y direction (FOV Y).

The method 204 yet further includes obtaining limits on adjustments of scan time and one or more image quality parameters for the scan protocol (block 216). The limits are site-specific limits. These site-specific limits may be part of the selected protocol or obtained separately from a database (e.g., database 184 in FIG. 2). These limits serve as rules to maintain constraints over scan time and certain image quality parameters (e.g., signal-to-noise ratio) while maintaining the contrast-to-noise ratio in the acquired image. In certain embodiments, the contrast is kept constant but the noise may be minimally adjusted, thus, slightly adjusting the contrast-to-noise ratio (but keeping it within a desired limit). These limits may include ranges for the scan time or image quality parameters (e.g., plus or minus a certain percent). In certain embodiments, both the signal-to-noise ratio and the contrast-to-noise ratio may be maintained It should be noted that although image quality is determined by signal-to-noise ratio and contrast-to noise ratio, other parameters may be utilized for determining image quality. In certain embodiments, the limits on adjustments are based on a condition of the subject. The limits on adjustments is obtained by the third module 194 of the prescription engine 188 in FIG. 3.

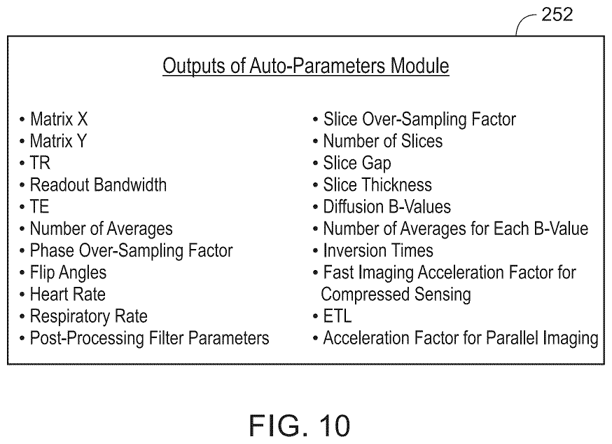

The method 204 still further includes generating an updated scan protocol by automatically adjusting one or more parameters (e.g., prescription parameters) of the scan protocol based on the scan protocol, the limits on adjustments, and the coverage of the scan (block 218). The updated scan protocol is generated utilizing the third module 194 of the prescription engine 188 in FIG. 3. The third module 194 in generating the updated scan protocol may output one or more of the following of prescription parameters: matrix size in the X direction (MatrixX), matrix size in the Y direction (MatrixY), echo train length, and an acceleration factor for parallel imaging. These prescription parameters may affect image quality. It should be noted that the prescription parameters that may be adjusted are not limited to these parameters. FIG. 10 provides a list (e.g., non-exhaustive and non-limiting list) of additional prescription parameters that may be outputted/adjusted. The method 204 further includes initiating the scan (e.g., diagnostic scan) of the subject with MRI system utilizing the updated scan protocol (block 220). The updated scan protocol maintains the image quality of the acquired image with respect to a baseline protocol (i.e., the selected scan protocol). Maintaining image quality keeps the image quality consistent with a desired site-specific image quality level. The image quality is maintained (e.g., consistent) between all scans at a site across all of the different MRI systems for the site for a given scan protocol. In certain embodiments, the contrast-to-noise ratio is maintained. In certain embodiments, the contrast is kept constant but the noise may be minimally adjusted, thus, slightly adjusting the contrast-to-noise ratio (but keeping it within a desired limit). In certain embodiments, both the signal-to-noise ratio and the contrast-to-noise ratio is maintained.

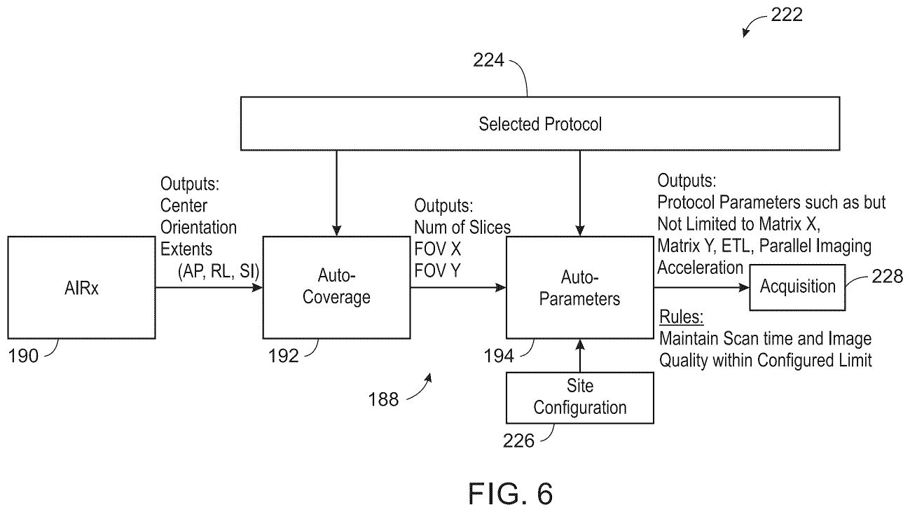

FIG. 6 illustrates a schematic diagram of a workflow 222 for performing a scan of a patient. The workflow 222 includes utilizing module 190 (e.g., AIRₓ) of the prescription engine 188 to automatically detect the anatomic landmark of interest in localizer images and determining a geometry plan (e.g., prescribed slices including center and orientation) of the scan of the anatomic landmark of interest including extents (e.g., AP. RL. SI) of the anatomic landmark of interest of the subject based on the localizer images. The localizer images are a series of images (e.g., two-dimensional (2D) images) in the axial, sagittal, and coronal planes (e.g., 3-plane localizer scans) obtained from a scout image of the subject. The module 190 outputs the extents of the anatomic landmark of interest. The module 190 also outputs a center orientation.

As depicted in the workflow 222, the module 192 (e.g., auto-coverage module) of the prescription engine 188 receives both the outputs of the module 190 and a selected protocol 224 for the scan of the subject. The module 192 automatically determines a coverage of the scan to include the anatomic landmark of interest and to match the extents of the anatomic landmark of interest based on the scan protocol and the geometry plan, including the extents of the anatomic landmark of interest. The determined coverage restricts the scan to the anatomic landmark of interest and avoids scanning unwanted regions. The module 192 determines and outputs one or more a number of slices, a field of view in the X direction (FOV X), and a field of view in the Y direction (FOV Y).

As depicted in the workflow 222, the module 194 (e.g., auto-parameters module) of the prescription engine 188 receives both the outputs of the module 192 and the selected protocol 224 for the scan of the subject. The module 194 also obtains limits 226 (e.g., site configuration) on adjustments of scan time and one or more image quality parameters for the scan protocol 224. The limits are site-specific limits. These site-specific limits may be part of the selected protocol 224 or obtained separately from a database (e.g., database 184 in FIG. 2). These limits serve as rules to maintain constraints over scan time and certain image quality parameters (e.g., signal-to-noise ratio) while maintaining the contrast-to-noise ratio in the acquired image. In certain embodiments, the contrast is kept constant but the noise may be minimally adjusted, thus, slightly adjusting the contrast-to-noise ratio (but keeping it within a desired limit). These limits may include ranges for the scan time or image quality parameters (e.g., plus or minus a certain percent). In certain embodiments, both the signal-to-noise ratio and the contrast-to-noise ratio may be maintained It should be noted that although image quality is determined by signal-to-noise ratio and contrast-to noise ratio, other parameters may be utilized for determining image quality. In certain embodiments, the limits 226 on adjustments are based on a condition of the subject.

The module 194 generates an updated scan protocol by automatically adjusting one or more parameters (e.g., prescription parameters) of the scan protocol based on the scan protocol 224, the limits 226 on adjustments, and the coverage of the scan. The module 194 in generating the updated scan protocol may output one or more of the following of prescription parameters: matrix size in the X direction (MatrixX), matrix size in the Y direction (MatrixY), echo train length, and an acceleration factor for parallel imaging. These prescription parameters may affect image quality. It should be noted that the prescription parameters that may be adjusted are not limited to these parameters. These are only a few examples of prescription parameters. FIG. 10 provides a list (e.g., non-exhaustive and non-limiting list) of additional prescription parameters that may be outputted/adjusted.

As depicted in the workflow 222, upon updating the scan protocol 224, the scan (e.g., diagnostic scan) of the subject with MRI system is initiated utilizing the updated scan protocol (as indicated by reference numeral 228). The updated scan protocol maintains the image quality of the acquired image with respect to a baseline protocol (i.e., the selected scan protocol). Maintaining image quality keeps the image quality consistent with a desired site-specific image quality level. The image quality is maintained (e.g., consistent) between all scans at a site across all of the different MRI systems for the site for a given scan protocol. In certain embodiments, the contrast-to-noise ratio is maintained. In certain embodiments, the contrast is kept constant but the noise may be minimally adjusted, thus, slightly adjusting the contrast-to-noise ratio (but keeping it within a desired limit). In certain embodiments, both the signal-to-noise ratio and the contrast-to-noise ratio is maintained.

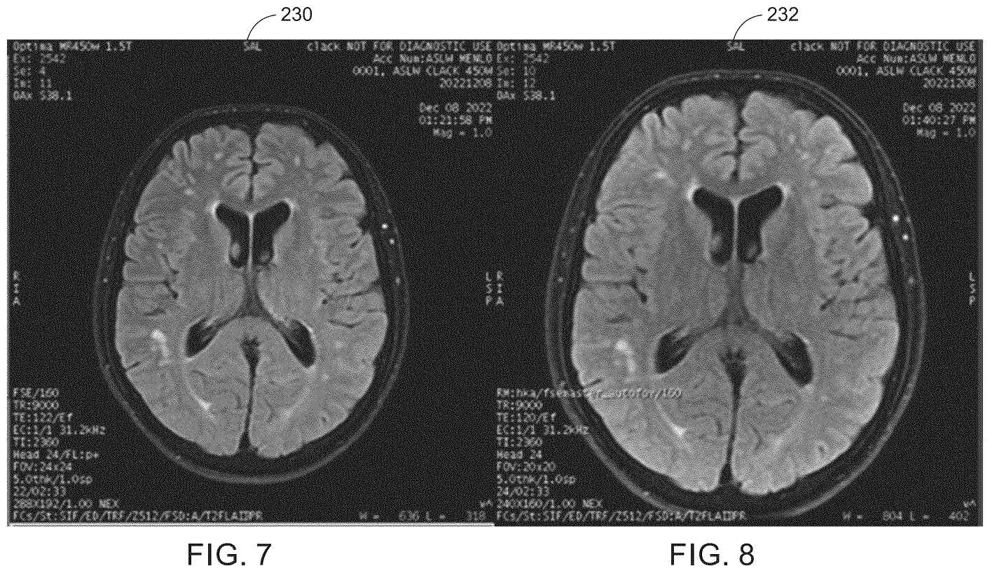

FIG. 7 is an MR image 230 obtained of an anatomic landmark of interest (e.g., brain) of a subject. FIG. 8 is also an MR image 232 obtained of an anatomic landmark of interest (e.g., brain) of a subject. Image 230 was obtained utilizing the correct orientation and center. However, acquisition of image 230 did not take into account a size of the patient, thus, a default protocol for FOV/coverage was utilized. Image 232 was obtained utilizing an update protocol from the prescription engine 188 described above. In particular, the image 232 was obtained with the correct orientation and center. In addition, the image 232 was obtained utilizing a FOV and number of slices reduced to match the head size of the subject. In particular, the prescription engine 188 adapted to the subject but maintained the desired image quality for the site.

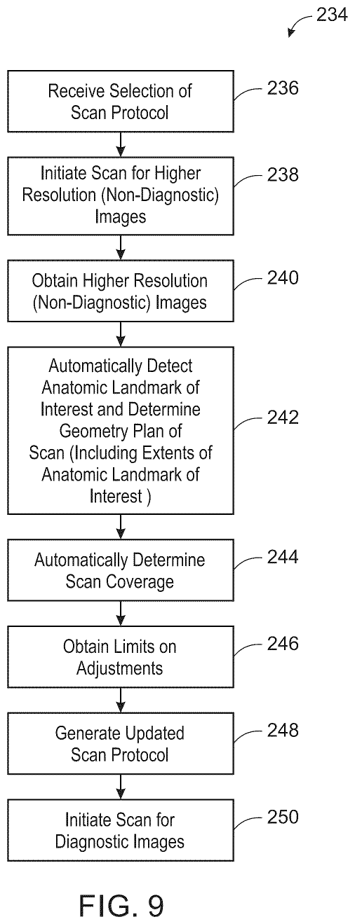

FIG. 9 illustrates a flow diagram of a method 234 for performing a scan of a patient utilizing the MRI system in FIG. 1 (e.g., utilizing higher resolution images instead of scout or localizer images). One or more steps of the method 204 may be performed by processing circuitry of the magnetic resonance imaging system 100 in FIG. 1. One or more of the steps of the method 234 may be performed simultaneously or in a different order from the order depicted in FIG. 9.

The method 234 includes receiving a selection of a scan protocol from among a plurality of scan protocols for a scan of a subject (e.g., patient) with an MRI system (e.g., MRI system 100 in FIG. 1) (block 236). The scan protocol serves as a baseline protocol. The method 234 also includes initiating a scan (e.g., non-diagnostic scan) of the subject with the MRI system to obtain higher resolution images (but non-diagnostic images) (block 238). The higher resolution images have a higher resolution than scout and localizer images. The method 234 further includes obtaining the higher resolution images including an anatomic landmark of interest of the subject (block 240).

The method 234 still further includes automatically detecting the anatomic landmark of interest in localizer images and determining a geometry plan (e.g., prescribed slices including center and orientation) of the scan of the anatomic landmark of interest based on the higher resolution images (block 242) Determining the geometry plan (block 242) also includes determining extents (e.g., AP. RL, SI) of the anatomic landmark of interest of the subject. The first module 190 of the prescription engine 188 in FIG. 3 is utilized for block 242 of the method 234. The first module 190 also determines as part of the geometry plan a center and orientation. The first module 190 further determines landmarks of the anatomic landmark of interest.

The method 234 even further includes automatically determining a coverage of the scan to include the anatomic landmark of interest and to match the extents of the anatomic landmark of interest based on the scan protocol, the geometry plan, and the extents of the anatomic landmark of interest (block 244). The determined coverage restricts the scan to the anatomic landmark of interest and avoids scanning unwanted regions. The second module 192 of the prescription engine 188 in FIG. 3 is utilized for block 244 of the method 234. Determining the coverage includes determining and outputting one or more a number of slices, a field of view in the X direction (FOV X), and a field of view in the Y direction (FOV Y).

The method 234 yet further includes obtaining limits on adjustments of scan time and one or more image quality parameters for the scan protocol (block 246). The limits are site-specific limits. These site-specific limits may be part of the selected protocol or obtained separately from a database (e.g., database 184 in FIG. 2). These limits serve as rules to maintain constraints over scan time and certain image quality parameters (e.g., signal-to-noise ratio) while maintaining the contrast-to-noise ratio in the acquired image. In certain embodiments, the contrast is kept constant but the noise may be minimally adjusted, thus, slightly adjusting the contrast-to-noise ratio (but keeping it within a desired limit). These limits may include ranges for the scan time or image quality parameters (e.g., plus or minus a certain percent). In certain embodiments, both the signal-to-noise ratio and the contrast-to-noise ratio may be maintained. It should be noted that although image quality is determined by signal-to-noise ratio and contrast-to noise ratio, other parameters may be utilized for determining image quality. In certain embodiments, the limits on adjustments are based on a condition of the subject. The limits on adjustments is obtained by the third module 194 of the prescription engine 188 in FIG. 3.

The method 234 still further includes generating an updated scan protocol by automatically adjusting one or more parameters (e.g., prescription parameters) of the scan protocol based on the scan protocol, the limits on adjustments, and the coverage of the scan (block 248). The updated scan protocol is generated utilizing the third module 194 of the prescription engine 188 in FIG. 3. The third module 194 in generating the updated scan protocol may output one or more of the following of prescription parameters: matrix size in the X direction (MatrixX), matrix size in the Y direction (MatrixY), echo train length, and an acceleration factor for parallel imaging. These prescription parameters may affect image quality. It should be noted that the prescription parameters that may be adjusted are not limited to these parameters. FIG. 10 provides a list (e.g., non-exhaustive and non-limiting list) of additional prescription parameters that may be outputted/adjusted. The method 234 further includes initiating the scan (e.g., diagnostic scan) of the subject with MRI system utilizing the updated scan protocol (block 250). The updated scan protocol maintains the image quality of the acquired image with respect to a baseline protocol (i.e., the selected scan protocol). Maintaining image quality keeps the image quality consistent with a desired site-specific image quality level. The image quality is maintained (e.g., consistent) between all scans at a site across all of the different MRI systems for the site for a given scan protocol. In certain embodiments, the contrast-to-noise ratio is maintained. In certain embodiments, the contrast is kept constant but the noise may be minimally adjusted, thus, slightly adjusting the contrast-to-noise ratio (but keeping it within a desired limit). In certain embodiments, both the signal-to-noise ratio and the contrast-to-noise ratio is maintained.

FIG. 10 is a schematic diagram illustrating outputs of an auto-parameters module. In particular, FIG. 10 includes a table 252 listing the outputs of the third module 194 (e.g., auto-parameters module) in FIG. 3. The outputs are prescription parameters that may be adjusted by the third module 194. The table252 is non-exhaustive and non-limiting list of prescription parameters that be adjusted. As depicted in the table 252, the outputs may include matrixX, matrix Y, repetition time (TR), readout bandwidth, echo time (TE), number of averages, phase over-sampling factor, flip angles, heart rate, respiratory rate, post-processing filter parameters, slice over-sampling factor number of slices, slice gap, slice thickness, diffusion B-values, number of averages for each B-value, inversion times, and fast imaging acceleration for compressed sensing, echo train length (ETL), and acceleration factor for parallel imaging.

Technical effects of the disclosed subject matter include providing utilization of a prescription engine that utilizes patient size-related constraints while ensuring image quality does not differ from baseline protocol within site-specified limits. Technical effect of the disclosed subject matter further include restricting a scan to an anatomy of interest and avoiding scanning unwanted regions. Technical effects of the disclosed subject matter also include providing optimal image quality irrespective of patient size changes and ensure reduced scan time. Technical effects of the disclosed subject matter further include enabling wider acceptance of intelligent prescription. Technical effects of the disclosed subject matter even further include less dependence on an MR technologist's expertise to obtain quality MR mages at an MR center (i.e., avoiding the interaction of the technologist to update prescription parameters to optimize image quality and scan time). Technical effects of the disclosed subject yet further include providing consistent MR images of good quality across an MR scanner for a patient. Technical effects of the disclosed subject matter even further include enabling a site to configure the ranges of the parameter adjustments to their preference.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing a scan of a subject utilizing a magnetic resonance imaging (MRI) system, comprising:
receiving, at a processor, a selection of a scan protocol from among a plurality of scan protocols for the scan of the subject with the MRI system;
obtaining, at the processor, localizer images including an anatomic landmark of interest of the subject acquired with the MRI system;
automatically detecting, via the processor, the anatomic landmark of interest in the localizer images and determining a geometry plan of the scan of the anatomic landmark of interest including extents of the anatomic landmark of interest of the subject based on the localizer images;
automatically determining, via the processor, a coverage of the scan to include the anatomic landmark of interest and to match the extents of the anatomic landmark of interest based on the scan protocol and the geometry plan including the extents of the anatomic landmark of interest;
obtaining, at the processor, limits on adjustments of scan time and one or more image quality parameters for the scan protocol; and
generating, via the processor, an updated scan protocol by automatically adjusting one or more parameters of the scan protocol based on the scan protocol, the limits on adjustments, and the coverage of the scan.

2. The computer-implemented method of claim 1, further comprising initiating, via the processor, the scan of the subject utilizing the updated scan protocol.

3. The computer-implemented method of claim 2, wherein the updated scan protocol maintains an image quality of an acquired image obtained from the scan relative to the scan protocol.

4. The computer-implemented method of claim 3, wherein maintaining the image quality comprises maintaining a contrast-to-noise ratio in the acquired image.

5. The computer-implemented method of claim 4, wherein the one or more adjusted parameters comprise a signal-to-noise ratio.

6. The computer-implemented method of claim 3, wherein maintaining the image quality comprises maintaining both a signal-to-noise ratio and a contrast-to-noise ratio in the acquired image.

7. The computer-implemented method of claim 1, wherein the limits on adjustments apply to use of all MRI systems at a site having one or more MRI systems including the MRI system or to use of all MRI systems across a network having multiple MRI systems across multiple sites including the MRI system.

8. The computer-implemented method of claim 1, wherein the limits on adjustments are based on a condition of the subject.

9. The computer-implemented method of claim 1, wherein the one or more adjusted parameters comprise one or more of matrix size, echo train length, an acceleration factor for parallel imaging, repetition time, readout bandwidth, echo time, number of averages, phase over-sampling factor, flip angles, heart rate, respiratory rate, post-processing filter parameters, slice over-sampling factor number of slices, slice gap, slice thickness, diffusion B-values, number of averages for each B-value, inversion times, and fast imaging acceleration for compressed sensing.

10. A system for performing a scan of a subject utilizing a magnetic resonance imaging (MRI) system, comprising:
a memory encoding processor-executable routines; and
a processor configured to access the memory and to execute the processor-executable routines, wherein the processor-executable routines, when executed by the processor, cause the processor to:
receive a selection of a scan protocol from among a plurality of scan protocols for the scan of the subject with the MRI system;
obtain localizer images including an anatomic landmark of interest of the subject acquired with the MRI system;
automatically detect the anatomic landmark of interest in the localizer images and determining a geometry plan of the scan of the anatomic landmark of interest including extents of the anatomic landmark of interest of the subject based on the localizer images;
automatically determine a coverage of the scan to include the anatomic landmark of interest and to match the extents of the anatomic landmark of interest based on the scan protocol and the geometry plan including the extents of the anatomic landmark of interest;
obtain limits on adjustments of scan time and one or more image quality parameters for the scan protocol; and
generate an updated scan protocol by automatically adjusting one or more parameters of the scan protocol based on the scan protocol, the limits on adjustments, and the coverage of the scan.

11. The system of claim 10, wherein the processor-executable routines, when executed by the processor, further cause the processor to initiate the scan of the subject utilizing the updated scan protocol.

12. The system of claim 11, wherein the updated scan protocol maintains an image quality of an acquired image obtained from the scan relative to the scan protocol.

13. The system of claim 12, wherein maintaining the image quality comprises maintaining a contrast-to-noise ratio in the acquired image.

14. The system of claim 13, wherein the one or more adjusted parameters comprise a signal-to-noise ratio.

15. The system of claim 12, wherein maintaining the image quality comprises maintaining both a signal-to-noise ratio and a contrast-to-noise ratio in the acquired image.

16. The system of claim 10, wherein the limits on adjustments apply to use of all MRI systems at a site having one or more MRI systems including the MRI system or to use of all MRI systems across a network having multiple MRI systems across multiple sites including the MRI system.

17. The system of claim 10, wherein the limits on adjustments are based on a condition of the subject.

18. The system of claim 10, wherein the one or more adjusted parameters comprise one or more of matrix size, echo train length, an acceleration factor for parallel imaging, repetition time, readout bandwidth, echo time, number of averages, phase over-sampling factor, flip angles, heart rate, respiratory rate, post-processing filter parameters, slice over-sampling factor number of slices, slice gap, slice thickness, diffusion B-values, number of averages for each B-value, inversion times, and fast imaging acceleration for compressed sensing.

19. A non-transitory computer-readable medium, the computer-readable medium comprising processor-executable code that when executed by a processor, causes the processor to:
receive a selection of a scan protocol from among a plurality of scan protocols for a scan of a subject with a magnetic resonance imaging (MRI) system;
obtain images including an anatomic landmark of interest of the subject acquired with the MRI system, wherein the images have a higher resolution than localizer images;
automatically detect the anatomic landmark of interest in the images and determining a geometry plan of the scan of the anatomic landmark of interest including extents of the anatomic landmark of interest of the subject based on the images;
automatically determine a coverage of the scan to include the anatomic landmark of interest and to match the extents of the anatomic landmark of interest based on the scan protocol and the geometry plan including the extents of the anatomic landmark of interest;
obtain limits on adjustments of scan time and one or more image quality parameters for the scan protocol; and
generate an updated scan protocol by automatically adjusting one or more parameters of the scan protocol based on the scan protocol, the limits on adjustments, and the coverage of the scan.

20. The non-transitory computer-readable medium of claim 19, wherein the processor-executable code, when executed by the processor, further cause the processor to initiate the scan of the subject utilizing the updated scan protocol, and wherein the updated scan protocol maintains a contrast-to-noise ratio of an acquired image obtained from the scan relative to the scan protocol.
